(19) United States
(12) Patent Application Publication
(10) Pub. No.: US 2005/0085534 A1
(43) Pub. Date: Apr. 21, 2005

(54) Process for the preparation of citalopram

(76) Inventors: Sujay Biwas, Kalinganj (IN); Tarun Kant Sharma, Muzaffarpur (IN); Yatendra Kumar, Gurgaon (IN); Swargam Sathyanarayana, Karim Nage (IN); Bakthavathsalan Vijayaraghavan, Guraon (IN)

Correspondence Address: Jayadeep R Deshmukh;Ranbaxy Pharmaceuticals Inc, 600 College Road East, Suite 2100, Princeton, NJ, 08540, US

(21) Appl. No.: 10/469,329

(22) PCT Filed: Mar. 8, 2002
(86) PCT No.: PCT/IB02/00690

(30) Foreign Application Priority Data
Mar. 9, 2001 (IN) 264DEL2001

(51) Int. Cl. C07D 307/87; A61K 31/343

(52) U.S. Cl. 514/469; 549/467

(57) ABSTRACT

The present invention relates to an improved and industrially advantageous process for the preparation of citalopram represented by the following Formula I, and pharmaceutically acceptable acid addition salt thereof.

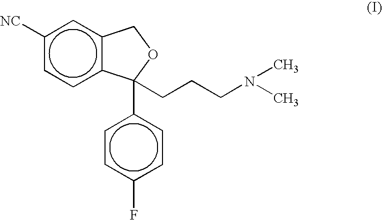

### FIELD OF THE INVENTION

The present invention relates to an improved and industrially advantageous process for the preparation of citalopram represented by the following Formula I, and pharmaceutically acceptable acid addition salts thereof.

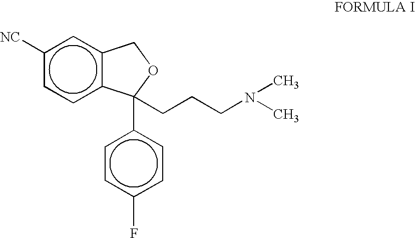

### BACKGROUND OF THE INVENTION

Citalopram is a well known anti-depressant drug and is chemically known as 1-[3-(dimethylamino)propyl]-1-(4-fluorophenyl)-1,3-dihydro-5-isobenzofurancarbonitrile. It is a selective centrally acting serotonin (5-hydroxy-tryptamine; 5-HT) re-uptake inhibitor and was described for the first time in U.S. Pat. No. 4,136,193. Citalopram is further used in the treatment of dementia and cerebrovascular disorders as disclosed in European Patent No. 474,580.

A method for preparing citalopram is described in U.S. Pat. No. 4,136,193. According to the invention, 4-halo-2-(hydroxymethyl)phenyl-(4′-fluorophenyl)-(3-dimethylaminopropyl)methanol represented by the following Formula II, 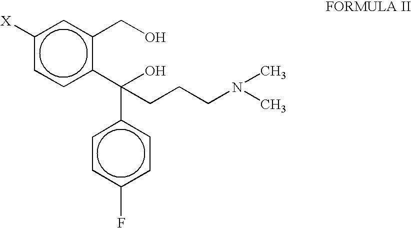
wherein X represents halogen, is reacted with a dehydrating agent to effect ring closure for obtaining 5-halophthalane compound represented by the following Formula III, 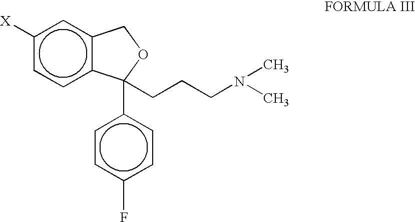
wherein X represents halogen. The compound of Formula III is reacted with cuprous cyanide in an inert organic solvent to give citalopram of Formula I. However, the process is unsuitable for accomplishment on an industrial scale since exchange reaction of the 5-halophthalane compound and cuprous cyanide does not go to completion even after refluxing them overnight in dimethylformamide thereby making it very difficult to separate the resulting citalopram from the corresponding 5-halo compound.

WO 00/13648 discloses the preparation of citalopram by reacting the 5-halophthalane compound of Formula III wherein X is bromo or iodo or the corresponding triflate compound with a cyanide source in the presence of a palladium catalyst and a catalytic amount of Cu⁺ or Zn²⁺ or with zinc cyanide in the presence of a palladium catalyst, and isolation of the corresponding 5-cyano phthalane compound i.e. citalopram. The cyanide source is chosen from potassium cyanide, sodium cyanide, ammonium cyanide and tetra alkyl ammonium cyanide.

A variant of this process is described in another PCT application, WO 00/11926, wherein the cyanide exchange is achieved with a cyanide source in the presence of a nickel catalyst.

The processes described in the above PCT applications for the manufacture of citalopram suffer from the following limitations and for various reasons stated below are not suitable for commercial purposes.
The reaction is carried out in the presence of palladium or nickel complexes which are very expensive, inconvenient to handle at commercial scale as they are air sensitive and light sensitive, highly flammable, cancer suspect agents and have limited commercial availability.
The reaction conditions are unsafe and are burdened with the risk of explosion and fire as the processes make use of solvents like tetrahydrofuran and diethyl ether.

Another process described in PCT application WO 01/02383 comprises the conversion of 5-halophthalane of Formula III to the corresponding Grignard reagent which is then converted to citalopram via reaction with compounds containing a cyano group bound to a leaving group. An alternative process involves obtaining an aldehyde from the Grignard reagent and its transformation to cyano group via an oxime or hydrazone intermediate.

The process described in WO 01/02383 involves many steps and make use of raw materials which are not available commercially.

Accordingly, none of the processes described heretofore are completely satisfactory at a commercial scale.

### SUMMARY OF THE INVENTION

It is an object of the present invention to solve the problems associated with the prior art and to provide an efficient and commercially viable process for producing citalopram via an improved cyano exchange process. The process is simple and provides obvious benefits with respect to economics and convenience to operate at a commercial scale.

More particularly, the present invention relates to a process for the preparation of citalopram of Formula I 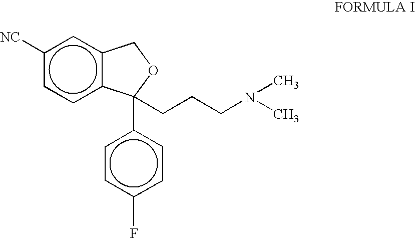
comprising reacting 5-halophthalane compound of Formula III, 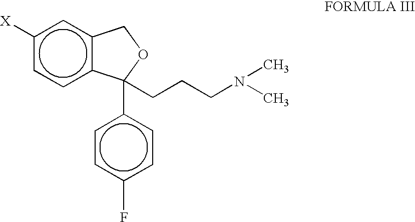
wherein X is bromo or iodo with a cyanide source in a suitable solvent, in the presence of an organic base and isolating corresponding 5-cyano compound i.e. citalopram of Formula I as the free base or in the form of a pharmaceutically acceptable acid addition salt thereof.

In a further aspect the invention relates to the above process which produces S-enantiomer of Formula I.

The cyanide source may be any source which is a cyanide ion donor. Preferred sources are potassium cyanide, sodium cyanide, ammonium cyanide, cuprous cyanide, zinc cyanide, tetra-alkylammonium cyanide or mixtures thereof. More preferred sources are cuprous cyanide and zinc cyanide. The cyanide source may be used in stoichiometric amount or in excess. Preferably, 1 to 2 molar equivalents per equivalent of compound of Formula III is used.

The term “suitable solvent” means any polar aprotic solvent. Preferably, the solvent may be selected from the group consisting of dimethylformamide, dimethylacetamide, N-methylpyrrolidone, N-methylpiperidinone, 1,3-dimethyl-3,4,5,6-tetrahydro(2H) pyrimidinone (DMPU), or mixtures thereof.

Suitable organic base includes trimethylamine, triethylamine, diisopropylamine, picolines, pyridine, pyridine derivatives such as 2,6-lutidine, 4-methylpyridine morpholine, morpholine derivatives, quinoline, 1,8-diazabicyclo[5.4.0] undec-7-ene (DBU), piperidine, aryl substituted amines such as aniline and dicyclohexylamine, or mixtures thereof. Preferably, pyridine or quinoline is used. The organic base may be used in stoichiometric amount or in excess. Preferably, about 1 to 5 molar equivalents per equivalent of starting material of Formula III is used.

We believe that nitrogen containing organic base plays a crucial role and facilitates the completion of reaction. The base is believed to form a complex of Formula IV in case of cuprous cyanide, 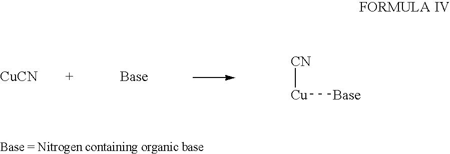
with the cyanide source which facilitates the exchange of halogen with nitrile via a transient state which involves a coordination complex of formula V, 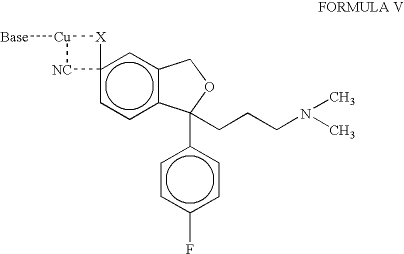
The reaction is generally carried out at a temperature ranging from about 120° C. to 170° C., preferably, at 135° C. to 145° C. The reaction completion may take from about 3 hours to several hours.

The intermediate of Formula III wherein X is bromo or iodo may be prepared from bromo or iodophthalide respectively, as described in U.S. Pat. No. 4,136,193, which is hereby incorporated herein by reference.

Citalopram of Formula I may be obtained as the free base or converted into its pharmaceutically acceptable acid addition salts. Examples of such salts include those formed with organic acids such as maleic, fumaric, benzoic, ascorbic, succinic, oxalic, bismethylenesalicylic, methanesulfonic, ethanedisulfonic, acetic, propionic, tartaric, salicylic, citric, gluconic, lactic, malic, mandelic, cinnamic, aspartic, stearic, palmitic, itaconic, glycolic, glutamic and benzene sulfonic acids or with inorganic acids such as hydrochloric, hydrobromic, sulfuric, sulfamic, phosphoric and nitric acid.

The acid addition salts of the compounds may be prepared by methods known in the art. The base is reacted with either the calculated amount of acid in a water miscible solvent such as ethanol or acetone and the salt is isolated after concentration and cooling or with an excess of the acid in a water immiscible solvent such as ether, dichloromethane or toluene with the salt separating out spontaneously.

### DETAILED DESCRIPTION OF THE INVENTION

The invention is further illustrated by the following example which should not be construed to be limiting the scope of the present invention.

### EXAMPLE 1

### Preparation of Citalopram Base

1-(4′-Fluorophenyl)-1-(3-dimethylaminopropyl)-5-iodophthalane (7.5 g, 18 mmol), cuprous cyanide powder (2.4 g, 27 mmol) and pyridine (5.6 g, 71 mmol) were added to dimethylformamide (40ml) and the mixture so obtained was heated to 140-141° C. The reaction mixture was further stirred at 140-145° C. for about 3 hours. The reaction mixture was then cooled to 35° C., and diluted with a cooled mixture of toluene and water. The organic layer was separated, washed with ammonia solution and water. The toluene was recovered completely under vacuum to get the product as a free base in the form of an oil (6.0 g)

### EXAMPLE 2

### Preparation of Citalopram Hydrobromide

Toluene (40 ml) was added to the above obtained free base of citalopram (6.0 g) and stirred to obtain a homogeneous solution. To this solution, was added aqueous HBr solution (48%, 3.6 g). The reaction mixture so obtained was then stirred for about 4 hours at 5-10° C. and toluene layer was decanted off. Fresh toluene (40 ml) was added to it and further stirred at 5-10° C. The separated solid was filtered, washed with toluene and dried to obtain citalopram hydrobromide (6.7 g, yield 93.7%, purity >98,5% by HPLC) as a crystalline powder.

While the present invention has been described in terms of its specific embodiments, certain modifications and equivalents will be apparent to those skilled in the art and are intended to be included within the scope of the present invention.

1. A process for the preparation of citalopram of Formula I, 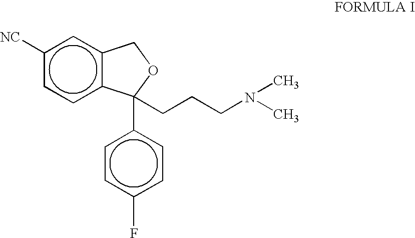
comprising reacting 5-halophthalane compound of Formula III, 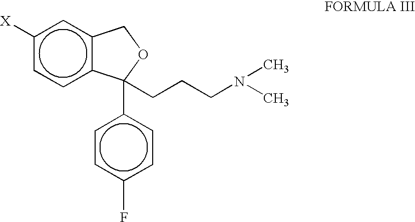
wherein X is bromo or iodo with a cyanide source in a suitable solvent in the presence of an organic base and isolating citalopram of Formula I, as the free base or in the form of a pharmaceutically acceptable acid addition salt thereof.

2. The process according to claim 1 wherein the cyanide source is any cyanide ion donor.

3. The process according to claim 2 wherein the cyanide ion donor is selected from the group consisting of potassium cyanide, sodium cyanide, ammonium cyanide, cuprous cyanide, zinc cyanide, ammonium cynide, tetra alkylammonium cyanide, and mixtures thereof.

4. The process according to claim 1 wherein the suitable solvent is a polar aprotic solvent.

5. The process according to claim 4 wherein the polar aprotic solvent is selected from the group consisting of dimethylformamide, dimethylacetamide, N-methylpyrrolidone, N-methylpiperidinone, 1,3-dimethyl-3,4,5,6-tetrahydro (2H) pyrimidinone (DMPU), and mixtures thereof.

6. The process according to claim 5 wherein the polar aprotic solvent is dimethylformamide.

7. The process according to claim 1 wherein the organic base is selected from the group consisting of trimethylamine, triethylamine, dilsopropylamine, picolines, pyridine, pyridine derivatives (wherein pyridine derivatives are 2,6-lutidine or 4-methyl pyridine), quinoline, 1,8-diazabicyclo [5.4.0] undec-7-ene (DBU), piperidine, aryl substituted amines (e.g. aniline), dicyclohexylamine, and mixtures thereof.

8. The process according to claim 7 wherein the organic base is pyridine or quinoline.

9. The process according to claim 7 wherein the organic base is used in stoichiometric amount or in excess ranging from about 1-5 molar equivalents per equivalent of the compound of Formula III.

10. The process according to claim 1 wherein the reaction is carried out at a temperature ranging from about 120° C. to 170° C.

11. The process according to claim 10 wherein the reaction is carried out at a temperature ranging from about 135 to 145° C.

12. The process according to claim 1 wherein the citalopram is isolated as the hydrobromide salt.
